(19) United States
(12) Patent Application Publication
(10) Pub. No.: US 2005/0197630 A1
(43) Pub. Date: Sep. 8, 2005

(54) Syringe handle

(76) Inventor: Ernst Sigurd Gustaf Folke Wiklund, Enskede Gard (SE)

Correspondence Address: WINSTON & STRAWN LLP, 1700 K STREET, N.W., WASHINGTON, DC, 20006, US

(21) Appl. No.: 11/122,501

(22) Filed: May 4, 2005

Related U.S. Application Data
(63) Continuation of application No. PCT/SE03/01763, filed on Nov. 17, 2003.

(30) Foreign Application Priority Data
Nov. 19, 2002 (SE) SE 0203440-3

(51) Int. Cl. A61M 5/00

(52) U.S. Cl. 604/239; 604/264

(57) ABSTRACT

Ergonomic syringe combination with a cannula body with a belonging plastic cannula and a needle body with a belonging puncture needle, which is surrounded by the plastic cannula and a grip, which allows a thumb/index finger grip close to and preferably a bit ahead of the point of the puncture needle, intended for the establishment of blood vessel access. The combination comprises two parts. One is a combination of a cannula body and a needle body where the needle body is surrounded by a tubular sleeve with an outgrowth used for mounting the other part. This one is a grip part with plates fitted over the outgrowth. The grip part can be rotated and angled in relation to the combination of cannula body and needle body and its puncture needle, to enable adaptation of the combination to each operator's own preferences with respect to right- respectively left-handedness and hand size.

### CROSS-REFERENCE TO RELATED APPLICATION

This application is a continuation of International application PCT/SE2003/001763 filed Nov. 17, 2003, the entire content of which is expressly incorporated herein by reference thereto.

### BACKGROUND ART

Medical care frequently requests the establishment of connections between blood vessels (veins and arteries) and injectors or containers of different kinds. The connection must be made with total sterility. Leakage must be avoided as far as possible. Examples of treatments requiring blood vessel access are blood transfusion, inclusive blood giving, blood sampling with the assistance of test containers under negative pressure, and intravenous supply of medicine, nourishment or just liquid for blood volume expansion, commonly called a drip. In all these applications it is essential that the puncture of the vessel wall can be done without damage to the surrounding tissues. Failures are unpleasant even under the best circumstances, but may cause serious damages as well.

A common syringe for insertion into blood vessels of different kinds is the so-called Dardel syringe, which was developed by chief surgeon Otto von Dardel at the anaesthesia department at Södersjukhuset at the end of 1960s. It became immediately a local success that gradually developed to global demand. Its advantages—when it has been put in place—are obvious. The global consumption is nowadays more than one thousand million units per year.

Unfortunately, the Dardel syringe is difficult to apply. Thus, it is often allowed to remain in place far too long. Nowadays the syringe use is not limited to anaesthesia departments, where the staff can maintain their ability by daily exercise. Thus, for a majority of certified medical staff failures are not uncommon—then the patient is told that he or she has “blood vessels that are exceptionally difficult to find”. To the patient a failed puncture attempt is no unimportant incident. Inter alia the patient gets a fair-sized “blue and black spot” but usually real pain is added as the blood vessel wall is well provided with nerves.

The Dardel syringe comprises a cannula body with a channel continuing with a thin, flexible, plastic tube. The rear opening of the cannula body is designed with a conical widening for connection to an injection pump or a catheter. The opening is from the beginning filled by the front part of the needle body, which continues forward with a tubular puncture needle, enclosed by the thin, flexible, plastic tube, and with a bevel cut, sharpened point, which ends some millimeters ahead of the thin, flexible, plastic tube's front end. The needle body has in its rear part a transparent signal space, which becomes filled by blood, when the blood path has been satisfactorily reached. The cannula body is provided with an upper port, which can be used for delivery of bolus doses of suitable medicines.

The Dardel syringe provides a very great step forward with respect to the patient's comfort at the set up of blood vessel accesses with a remaining cannula. Earlier rigid connections may be replaced with a soft cannula of a tissue compatible plastic.

When a blood vessel access is set up, one chooses a suitable superficial blood vessel (vein or artery, depending on the type of treatment) and tries to direct the point of the puncture needle for skin passage directly over the chosen blood vessel so the point hits the vessel centrally and after penetration of the vessel wall can, together with the plastic cannula, be directed axially obliquely into the blood path. The setting up of blood vessel accesses requests high accuracy.

When the mentioned signal space behind the needle starts filling with blood the needle body with its puncture needle is kept still, while the cannula body is advanced, so that the point of the needle is no longer ahead of the plastic cannula. This is done to prevent the needle point from hurting the vessel wall, when later the plastic cannula aided by the cannula body is advanced into the blood path to the main part of its length.

When the plastic cannula has arrived at desired position in the blood path, the cannula body is fixed on the skin. The puncture needle, which so far has prevented leakage by its close fit to the inner wall of the plastic tube, is withdrawn with the needle body and the desired connection to syringe, container or catheter is made.

For reasons of sterility the plastic tube must not be touched before or during the insertion into the blood lumen. The distance from the needle tip to the grip around the needle body is long. The grip surfaces are not well defined, especially as the hands most frequently have to be provided with gloves. The insertion demands a delicate row of complicated changes of direction. There is no visible indication of the position of the needle tip after the skin penetration. To this is added that the blood vessels are frequently relatively badly fixed under the skin and are having tendencies to “roll away”.

In spite of these very unfavorable circumstances from the ergonomic point of view, experienced nurses, with daily training, develop great skill and fail very seldom. The circumstances are different for doctors, nurses, ambulance crews and learners, which more temporarily are put to the task. Failures will cause the patient discomfort and are embarrassing to the operator. Besides loss of valuable material will arise.

The handling problems are caused mainly by the long distance between the needle point and the place where the operator grips the syringe. Anyone who has tried to write with a pencil kept at its rear end instead of near its point has probably noted how much wider the movements become. Besides the writing arm becomes tired much faster. From the ergonomic point of view a thumb/index finger grip near the needle point is much better.

Amendment proposals concerning the ergonomics at the establishment of vein and artery connections and similar operations can be found in British patent 1,416,432 and the German published application 4244563 and German Patent 4208228. The solution of the problem according to the former is applicable to injection needles without surrounding plastic tubes only. The latter one mentions the possibility of combination with a plastic tube.

A more complete proposal to amendment of the ergonomic conditions at the setting up of blood vessel accesses aided by a remaining cannula is presented in U.S. Pat. No. 6,197,006, which describes a handle for a syringe comprising a needle body with a tubular needle of suitable material and a cannula body with a thin, plastic cannula intended for the puncture of blood vessel walls and setting up blood path access. The handle is designed to admit placement of thumb and index finger at the side of the needle point and in its vicinity and prevents unintended position displacement between the needle body and the cannula body during the puncture phase. Even so, improvements in these type devices are desired and needed.

### SUMMARY OF THE INVENTION

The present invention refers to further development of the Dardel syringe to make it easier to handle for staff that does not regularly work with the setting up of blood vessel accesses. Also the invention uses the experiences obtained at the testing of mentioned handle according to U.S. Pat. No. 6,197,006.

Mentioned proposals to amend the ergonomics at setting up of blood vessel accesses have this in common that possibilities for adaptation to each operator own preferences are lacking. Doctors and other medical staff have different hand sizes. Even if a majority is right-handed, a substantial minority is left-handed. The present invention now resolves these problems.

The present invention specifically refers to a method to make the ergonomics at the setting up of blood vessel accesses adaptable to each operator's individual qualifications. To this effect the handle is made turnable with suitable intervals to fit right-or left-handed. Further possibilities are given for individual adaptation of the distance between the handle's front end and point of the puncture needle. By experience it has been shown that a grip part, admitting a thumb/index finger grip near to and preferably a bit ahead of the needle point, gives the best conditions from the ergonomic point of view.

Further, the invention refers to a new-designed syringe comprising two parts. In its turn one of these parts comprises a modified Dardel syringe, i.e., a cannula body with a flexible plastic cannula and a needle body with a puncture needle surrounded by the plastic cannula. The needle body is designed for comfortable mounting of the other part comprising a grip part, admitting placement of the operator's thumb/index finger grip so close to the needle point as the operator wishes. To permit this adaptation to the operator's individual preferences the grip part is adjustable for use by both right-handed and left-handed persons. Also, it can be angled to give suitable distance between the needle point and the grip point.

### BRIEF DESCRIPTION OF THE DRAWINGS

FIGS. 1a and b show a side view respectively an end view of a syringe according to a preferred embodiment of the invention.

FIGS. 2a and b show different sections through the needle body shown in FIG. 1.

FIG. 3 shows a side view of a cannula body with an end stopper.

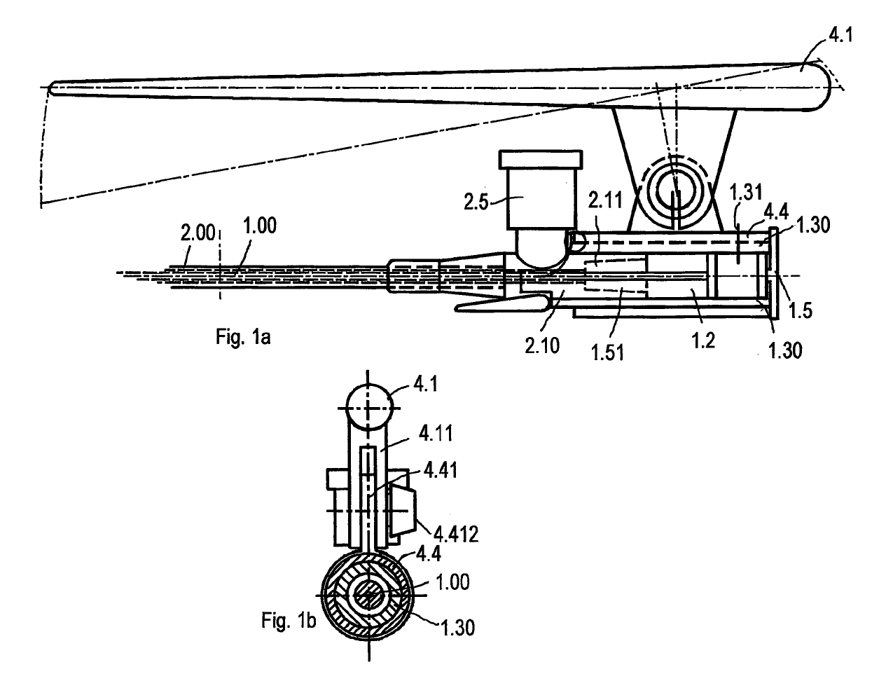

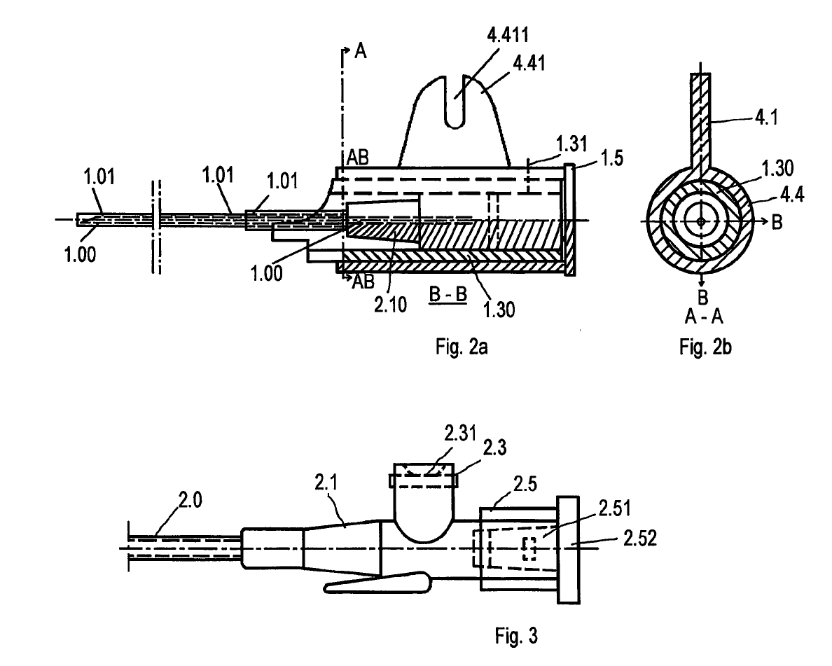

### DETAILED DESCRIPTION OF THE PREFERRED EMBODIMENTS

In one embodiment, the invention relates to an ergonomic syringe combination, comprising a cannula body that includes a cannula and a needle body that includes a puncture needle having a point intended for the establishment of blood vessel access, with the needle body surrounded by the cannula, a grip part which allows a thumb/index finger grip adjacent the point of the puncture needle, and two further parts, a sleeve surrounding the cannula body and needle body and including an outgrowth for mounting the grip part in a manner such that the grip part can be rotated and angled in relation to the cannula body, needle body and puncture needle to enable adaptation of the combination to each operator's own preferences with respect to right- and left-handedness and hand size.

Preferably, the grip part includes at least one plate, and more preferably two plates, operatively associated with the outgrowth for mounting of the same. Also, the outgrowth is advantageously provided with a slit to facilitate mounting of the grip part.

The numbering in the drawings is hierarchic, i.e. the figure before the point denotes if the detail belongs to the needle body (1), the cannula body (2) or the handle (4). In the following details special for the embodiment of the invention shown in the figures are elaborated.

The tube (4.4) makes up a socket for the grip part (4.1) via the plates (4.11) that straddle the turning tube's (4.4) outgrowth (4.41). The angle between the grip part (4.1) and the axis of the puncture needle is adjusted by means of a clamp-screw (4.412) that fixes the handle (4.1) in a desired angle to puncture needle and cannula. To make the mounting of the grip part (4.1) easier the outgrowth (4.41) should be provided with a slit (4.411) so that the clamp-screw (4.412) has to be loosened just some turns to allow the plates (4.11) to straddle the outgrowth (4.41). The tube (4.4) can be rotated round a stiffening tube (1.30) to adjust the grip part's (4.1) turning angle in relation to the upper port.

The outside of the tube's (4.4) front end may be provided with a scale that is read against an index at the upper port (2.3). This is in the figure shown by a circle. The interplay between an index at the upper port's neck may also be marked by notches in the terminal disk (1.5) in desired turning intervals in 30° steps.

The terminal disk (1.5) holds the edge of the tube (4.4) against the index with an elastic force. The rotation between the tube (4.4) and the stiffening tube (1.3) may be locked after final adjustment by the pressing in of a little peg (1.31), which in a suitable way has been fastened at the rear part of the tube (4.4) by pressing down the peg in the corresponding hole in (1.30).

The stiffening tube (1.30), which is fixed to the needle body, is thread on the rear part of the cannula body and has beside a desirable stabilizing finction as task to prevent rotation of the needle (1.00), when the tube (4.4) is turned. In this way it is safeguarded that the needle's eye is always kept upwards, which is essential to prevent unnecessary damages by cutting out tissue.

In the original version of the Dardel syringe the stopper (2.5) for the cannula body's rear end is temporarily placed in the rear end of the needle body. This has caused that it often follows the used needle. It is now according to U.S. Pat. No. 6,017,327, a document that is incorporated herein by reference. automatically left at the cannula body above the upper port. From there it may be taken with remaining sterility, when the needle has been removed from the cannula.

In a preferred embodiment of the invention, the upper port (2.3) is completely redesigned compared with the Dardel syringe. Instead of an upper connection, which is judged as a hygienic risk a self-sealing hygienic barrier (2.31) of a suitable elastomer intended to be punctured with a fine needle for deliverance of bolus doses is offered. The upper barrier surface becomes now easily available for wiping off immediately before each use.

As mentioned the new syringe construction comprises two parts: one syringe part and one grip part. The syringe part is intended for disposal after use and is delivered sterilely packed in the usual manner. As the operator wishes, it may be used with or without mounting of the grip part. This part does not need sterilization. Thus, it may be designed for repeated use.

The operator may be protected against stabs or scratches from the used needle by a point protector, which automatically is drawn or pushed forward some millimeters over the point, for instance according to U.S. Pat. No. 6,629,957, or by that the operator at the uncovering of the needle places the maker's protective cap at the mounted grip part either inside its rear end or as a temporary grip surface at the front end of the grip part. Then, in both cases, the drawn out needle may be provided with a robust protector in short time. If this is done the risk for damages in connection with the separation of the needle body and the grip part is eliminated.

1. Ergonomic syringe combination, comprising a cannula body that includes a cannula and a needle body that includes a puncture needle having a point intended for the establishment of blood vessel access, with the needle body surrounded by the cannula, a grip part which allows a thumb/index finger grip adjacent the point of the puncture needle, and two further parts, a sleeve surrounding the cannula body and needle body and including an outgrowth for mounting the grip part in a manner such that the grip part can be rotated and angled in relation to the cannula body, needle body and puncture needle to enable adaptation of the combination to each operator's own preferences with respect to right- and left-handedness and hand size.

2. The syringe combination of claim 1, wherein the grip part includes at least one plate operatively associated with the outgrowth for mounting of the same.

3. The syringe combination of claim 1, wherein the outgrowth includes an upstanding plate and the grip part includes two plates operatively associated with the outgrowth plate for mounting of the same.

4. The syringe combination of claim 1, wherein the outgrowth is provided with a slit to facilitate mounting of the grip part.

5. The syringe combination of claim 1, wherein the cannula body includes an upper port that is provided with a self-sealing hygiene barrier.

6. The syringe combination of claim 1, wherein the cannula body includes an upper port that is designed to carry a stopper for sealing the port when the needle has been withdrawn after puncture of a blood vessel.

7. The syringe combination of claim 1, wherein the cannula body includes an upper port that is provided with an index for reading against a scale carried by the tubular sleeve.

8. The syringe combination of claim 1, which further comprises a peg for engaging and locking the tubular sleeve outgrowth to prevent movement in relation to the needle body.

9. The syringe combination of claim 1, wherein the cannula is made of plastic.

10. The syringe combination of claim 1, wherein the sleeve is tubular.

11. The syringe combination of claim 1, wherein the tubular sleeve is made of plastic.

12. The syringe combination of claim 1, wherein the grip part extends slightly ahead of the point of the puncture needle.
